(19) United States
(12) Patent Application Publication
(10) Pub. No.: US 2005/0090545 A1
(43) Pub. Date: Apr. 28, 2005

(54) Nitric oxide topical technology

(76) Inventors: Lorn Leitman, Key Biscayne, FL (US); Gustavo Barni, Parkland, FL (US)

Correspondence Address: Lorn Leitman, 325 S. W. 15th Avenue, Pompano Beach, FL, 33069, US

(21) Appl. No.: 10/691,025

(22) Filed: Oct. 22, 2003

(51) Int. Cl. A61K 31/21; A61K 31/198

(52) U.S. Cl. 514/509; 514/565

(57) ABSTRACT

This invention with a new compound enables application of L-Arginine made with arginine alpha-ketoglutarate, arginine pyroglutamate, arginine ketoisocaproate and ornithine alpha-ketoglutarate to work together and separately along with other ingredients, topically. It is intended to improve the process by which these compounds work in the human organism. The invention is based on the facts that L-Arginine is: known to be the immediate precursor of the endogenous vasodilator substance found in the arterial blood vessels called “Endothelium-Derived Relaxing Factor” (EDRF) required for protein synthesis and, depending on the organism's needs, can either be metabolized to support glucose synthesis or catabolized to produce energy This simplified “noninvasive” application surpasses gastrointestinal digestion so the compounds will not degrade and favoring absorption into the circulatory system thus enhancing the compounds' effects and at much lower dosage levels than oral or parenteral administration. A composition comprising L-Arginine to be used topically that creates increases in muscle size, strength, endurance and power output.

### BACKGROUND OF THE INVENTION

1. Field of the Invention

The present invention relates to a new technology of a method of administration by which these main four (4) components arginine alpha-ketoglutarate, arginine pyroglutamate, arginine ketoisocaproate and ornithine alpha-ketoglutarate work together and separately topically. The topical applications are intended to improve the process by which these compounds work in the human organism.

2. Description of the Prior Art

Prior to this unique new technology of applying these components (arginine alpha-ketoglutarate, arginine pyroglutamate, arginine ketoisocaproate as well as ornithine alpha-ketoglutarate) topically, there are different combinations that have been used. In order to obtain virtually the same effect compared to what we have with the topical use, it was necessary to put these components in a tablet or capsule presentation and use a total of 6 grams (6,000 milligrams) to get any sort of effect for oral administration. With this new technology that we are providing only 1 gram (1,000 milligrams) of the application, which is almost 15% of what was needed before, is used to obtain a superior effect from the point of vasodilation effect, dilation of blood vessels, improvement in oxygen uptake, improvement in blood flow and circulation in a way that completely alternates this technology and the availability of the product. Plus the fact that with this topical presentation the components do not enter the liver and do not enter the digestive tract so it has many, many more benefits and powerful hemodilation properties which create dramatic increases in muscle size, strength and endurance in power output. All that is needed in this new and unique topical transdermal presentation is 1 gram (1,000 milligrams) for an improvement in:
vasodilation which enhances the blood flow
oxygen uptake which improves oxygenation
lactic acid reduction which enhances endurance and speeds recovery

This amount (1 gram) is far less than the predecessors which were in tablets and capsules and had to use 6 times as much components going through the digestive tract to get close to the benefits that the topical use can provide. 5 cc in a topical application is all that is needed for improvement in vasodilation.

### SUMMARY OF THE INVENTION

Indeed, the present inventor has found that topical applications of these components are useful for muscle stimulant in a human organism. This based on the fact that the parent compound, amino acid L-arginine, is required for protein synthesis and thus can either be metabolized to support glucose synthesis or catabolized to produce energy (depending on the needs of the organism) and it is also known to be the immediate precursor of the endogenous vasodilator substance found in the arterial blood vessels called “Endothelium-Derived Relaxing Factor” (EDRF).

The applications are for:
Insulin mimicking effect
Vasodilation effect or for better blood flow
Improvement in oxygen uptake
Enhance the production of the EDRF's improvement of blood flow and circulation.

As a result of its creating a powerful hemodilation effect this property creates dramatic increases in muscle size, strength, endurance and power output.

With its advanced topical technology the “Big 4” (arginine alpha-ketoglutarate, arginine pyroglutamate, arginine ketoisocaproate and omithine alpha-ketoglutarate) generate a virtual ongoing muscle pump. Through regular topical application of the “Big 4 you get hemodilation which means widening of the blood vessels leading to increased skeletal muscles through the mechanism of amplified blood flow. Additional benefits of the topical transdermal application of the “Big 4” (arginine alpha-ketoglutarate, arginine pyroglutamate, arginine ketoisocaproate and omithine alpha-ketoglutarate) is that they have the ability to help with a) impotence because they help to relax arteries in the penis allowing for better blood flow, b) wound healing because it increases protein synthesis, and c) endurance because to some degree good circulation is necessary to carry oxygen to muscles and metabolic wastes away from them. Application in the male organ it helps with dilation blood flow and sexual performance; application in the uterus in the female anatomy especially helps with older woman with better blood flow and increased lubrication.

Arginine alpha-ketoglutarate, arginine pyroglutamate, arginine ketoisocaproate as well as omithine alpha-ketoglutarate are all derived from L-arginine.

In addition Nitric Oxide (NO) is produced from L-arginine by the family of nitric oxide synthase (NOS) enzymes, forming the free radical NO and citrulline as by-products. Nitric oxide is an important bio-regulatory molecule in the nervous, immune and cardiovascular system. Nitric Oxide is synthesized from one of the guanidino nitrogens of L-arginine by the enzyme nitric oxide synthase (NOS). Since the discovery the endothelium derived relaxing factor (EDRF) by Furchgott and Zawadzki (Furchgott and Zawadzki, 1980) which was later identified as nitric oxide (NO) (Ignarro et al., 1987; Palmer et al., 1987, Furchgott, 1988) it has become clear that there are a number of additional endothelium-derived vasodilator and vasoconstrictor autacoids (endothelin-1, prostaglandin H2, and endothelium-derived hyperpolarizing factor: EDHF). None of these autacoids play such a central role in the regulation of vascular tone and homeostasis as the primary EDRF the free radical NO, which is generated via a live-electron oxidation of guanidino nitrogen from L-arginine by an NO synthase (NOS).

Alpha-ketoglutarate is a precursor of glutamine. The administration of alpha-ketoglutarate in combination with omithine improves gut morphology and functions, counteracts trauma-induced dysimmunity and exerts anabolic/anticatabolic actions on protein metabolism.

Also related to the protein metabolism, arginine load is extensively metabolized by the liver, inducing urea production. In relation with urea reduction a highly regulated expression of several enzymes present in the urea cycle occurs in many other tissues, where these enzymes are involved in synthesis of nitric oxide, polyamines, proline, and glutamate.

The generation of nitric oxide by the vascular endothelium maintains a continuous vasodilator tone that is essential for the regulation of blood flow and blood pressure.

In clinical investigations insulin stimulates both endothelin (ET-1) and nitric oxide (NO) activity in the human form; that is, the skeletal muscle circulation of insulin stimulates both ETI in nitric oxide activity.

Arginine pyroglutamate is an amino acid naturally found in vegetables, fruits, dairy products and meat. It is also present in large amounts in the human brain, cerebrospinal fluid and blood. No serious adverse effects from the use of pyroglutamate, or from the use of arginine pyroglutamate, have been reported. Arginine and pyroglutamate are amino acids found commonly in natural foods consumed by most people on a daily basis.

In the recent study, documented, in the British Journal of Pharmacology it was found that a component of isoprenaline-mediated vasorelaxation in pulmonary arteries is mediated by nitric oxide (NO). L-arginine uptake relaxation to the membrane permeant cyclic AMP analogue CPT cyclic AMP was also potentiated by L-arginine. What this means is that there was a greater relaxation and improvement in blood flow and therefore a stronger capacity in the lungs for oxygen and improvement in the ability of oxygenation of the blood in the rats. In concert, L-arginine is critically important for the development of the NO- (nitric oxide-) and endothelium-dependent component of cyclic AMP-induced vasorelaxation in rat pulmonary arteries.

### DETAILED DESCRIPTION OF THE PREFERRED EMBODIMENTS

L-Arginine is a non-essential amino acid which is abundant in protamines and histones, these associated with nucleic acids. L-Arginine is used by the immune system to help and regulate the activity of the thymus gland, which is responsible for manufacturing T-Lymphocytes. For this reason, it is very important nutrient for people suffering from AIDS and other malignant diseases which suppress the immune system. In the pancreas it is used to release insulin and in the pituitary gland is a component of human growth hormone, and is used in sexual stimulants, as people report longer and more intense orgasms when their intake of arginine is increased. It is important in liver health and assists in neutralizing ammonia in the liver, while it is also involved in the skin and connective tissue, making it important for the healing and repair of tissue as well as the formation of collagen and building of new bone and tendons. It is required in muscle metabolism and helping with weight control since it facilitated the increase of muscle mass, while reducing body fat. L-Arginine also creates nitric oxide in the human body (nitric oxide is one of the most essential substances which influence sexual functions in both men and women), and helps to improve the circulation and maintain the nitrogen balance. Arginine is found in seminal fluid and L-Arginine is used in the treatment of male sexual health and has been used in the treatment of sterility.

Sodium bicarbonate is referred to as an alkaline salt, meaning that it has the ability to neutralize or counteract acids. It is used in the formulation as an ergogenic aid, to combat the fatiguing effects of another acid, lactic acid. It aids in the formulation of this compound because it has the following effects: Reduces lactic acid accumulation, improves endurance performance, and increases power output.

Potassium Bicarbonate is a corrosion inhibitor and a convenient source of potassium It is also being used to prevent hypokalemia. Potassium concentration is of extreme importance since its level in the extracellular fluid (ECF) depends on the external (intake and output) and internal (distribution between extracellular and intracellular fluid—ICF—) balances. In the body there is a larger potassium concentration in the ICF pool so the ECF pool will fluctuate more dramatically with changes in total body potassium distribution. This distribution is affected by many factors (i.e. exercise and skin excretion by perspiration); strenuous exercise which may injure muscle cells and allow leakage of potassium into the ECF. However, highly physical training generally creates normal total body potassium content, but redistributes potassium into muscles which produces hypokalemia (a sign of potassium deficiency). Hypokalemia is known to cause muscle weakness and under extreme levels it is even capable of leading to paralysis. Under hypokalemia muscle membranes may be injured producing rhabdomyolysis (breakdown of muscle fibers with leakage of potentially toxic cellular contents into the systemic circulation) Hypokalemia suppresses insulin release leading to glucose intolerance and it can also cause intracellular acidosis.

Glycine is a nonessential amino acid used by the body to build proteins. Glycine may play a role in maintaining the health of the prostate. Glycine is used in our formulation because it helps in retarding degeneration of muscles since it helps to supply extra creatine in the body.

Isoporpyl Myristate is in the formula in order to obtain better absorption of the compound through the skin.

Distilled water is used in our formulation as a solvent for the above mentioned components.

Vitamin Skin smoother with corn in the form of cream is used in our formulation to use the cream as method of topical administration of the compound.

Suitable routes in the past included oral and parenteral administration. The oral dosage form had been preferred. However, these methods of administration had several disadvantages. Six times stronger compound had to be used, and such components had to go through the digestive tract in order to obtain the benefits that the topical compound can provide.

In order to overcome these disadvantages, the present inventor has discovered that is possible to obtain the benefits of this L-arginine compound by topically applying the compound to those desired areas. 5 cc of a topical application is all that is needed in order to obtain improvement in vasodilatation. 5 ml of cream must be applied 20 to 30 minutes prior to workout on body parts that are being trained. The compound may be used on one body part ( the part that is being trained that day). For example, if upper arms are being worked out, then 2.5 m are to be applied on each inner bicep. If legs are being worked out, 2.5 m are to be applied on each inner thigh.

By using this method of application, it has been found that the components of the compound do not enter the liver and do not enter the digestive track. However, it still has benefits and powerful hemodilation properties which create dramatic increases in muscle size, strength and endurance in power output.

A preferred formulation is set forth below:

| | |
|---|---|
| L-Arginine Alphaketoglutarate, dihydate | 1000 mg |
| Potassium Bicarbonate | 100 mg |
| Sodium Bicarbonate | 100 mg |
| Glycine | 100 mg |
| Isopropyl Myristate | 800 mg |
| Distilled Water | 700 mg |
| Vitamin Skin Smoother with Corn | 2000 mg |

The precise components of the formula set forth above are merely the preferred embodiments of the composition. Likewise, the weight value for each component is only the preferred value for the identified component.

The preferred composition of the present invention as identified above is exclusively in a topical form. The composition may be made by simply mixing and or blending the components together in the desired quantities. This procedure with the quantities mentioned in the formulation is used for the formulation of 600 bottles with 120 ml (4 ounces) per bottle.

### EXAMPLE 1

A preferred dosage is set forth below

| | Preferred Dosage |
|---|---|
| Compound | 5 ml |

The above composition was administered to test subjects. Those tests and the results are described in example 2.

### EXAMPLE 2

| Subject 1 | Subject 2 | Subject 3 |
|---|---|---|
| Gender: Male Subject | Male Subject | Male Subject |
| Age: 25 Years old | 43 Years old | 52 Years old |
| Weight: 239 lbs | 195 lbs | 225 lbs |

Dose: All subjects applied 5 ml of compound 20 to 30 minutes prior to work out on body parts that are being trained. Compound was applied on one body part ( upper arms, forearms, thighs, calved, chest, shoulders, and neck) per day, which was the body part being trained on that day. If upper arms were being worked out then 2.5 ml were applied on each inner bicep. If legs were being worked out then 2.5 ml were applied on each inner thigh.

Trial period: 30 days

REM: All subjects had 7 to 8 hours of sleep per day. Subject 1 took 100 mg 1-testosterone capsules, 4 per day during the complete treatment and modified his eating habits form day 1 to day 30 as follows: 6 meals per day ( 3 strong and 3 light meals per day during the 1^{st} week, 4 strong meal the 2^{nd} week and 3 strong meals thereafter. All these meals were low in carbohydrate content (the intention with this subject was to see if weight loss and improvement of his muscle cutting was feasible). Subjects 2 and 3 maintained their 3 strong meals with high carbohydrate content.

Result: Subject 1 reduced his weight from 238 lbs to 205 whereas subjects 2 and 3 retained their original body weight over the 30-day treatment period. After the 1^{st} week, the building muscle pump in the specific body part was sustained for about 72 hours. The muscle reflected an greatly improved and faster recuperation with no muscle pains. The body parts worked out were noticeable leaner, more cut with sustained muscle mass gain. All 3 subjects substantially increased their weightlifting capacity and repetitions leading to more strength/power output and endurance. Due to the fact that they were able to increase their workout intensity and there were more nutrients being carried through their blood to their muscle cells, the subjects had far less soreness and much greater recuperation of their muscles which in turn allowed for greater muscle growth. A sustained muscle pump was observed for periods lasting up to four days, which in fact could be measured with added circumference to the muscle. Over time, muscle gains and endurance became more permanent from progressive workouts accompanied with the 5 ml applications of the compound. While this invention has now been described with reference to several preferred embodiments, those skilled in the art will appreciate that various substitutions, omissions, modifications and changes may be made without departing from the scope or spirit thereof. Accordingly, it is intended that the foregoing description be considered merely exemplary of the invention, and not a limitation thereof.

1. A new method of improving the process by which a composition comprising L-Arginine can stimulate and work in human growth organism in need of increased muscle growth. An effective amount of the composition consisting essentially of:

| | |
|---|---|
| L-Arginine Alphaketoglutarate, dihydate | 1000 mg |
| Potassium Bicarbonate | 100 mg |
| Sodium Bicarbonate | 100 mg |
| Glycine | 100 mg |
| Isopropyl Myristate | 800 mg |
| Distilled Water | 700 mg |
| Vitamin Skin Smoother with Corn | 2000 mg |

2. The method as claimed in claim 1, wherein, the composition is administered topically.

3. A new method to improve muscle growth, endurance, strength and power output and to aid in fat depletion, said composition consisting essentially of:

| | |
|---|---|
| Arginine Pyroglutamate | 1000 mg |
| Potassium Bicarbonate | 100 mg |
| Sodium Bicarbonate | 100 mg |
| Glycine | 100 mg |
| Isopropyl Myristate | 800 mg |
| Distilled Water | 700 mg |
| Vitamin Skin Smoother with Corn | 2000 mg |

4. The method as claimed in claim 3, wherein, the composition is administered topically.

5. A new method to improve muscle growth, endurance, strength and power output and to aid in fat depletion, said composition consisting essentially of:

| | |
|---|---|
| Arginine Ketoisocaproate | 1000 mg |
| Potassium Bicarbonate | 100 mg |
| Sodium Bicarbonate | 100 mg |
| Glycine | 100 mg |
| Isopropyl Myristate | 800 mg |
| Distilled Water | 700 mg |
| Vitamin Skin Smoother with Corn | 2000 mg |

6. The method as claimed in claim 5, wherein, the composition is administered topically.

7. A new method to improve muscle growth, endurance, strength and power output and to aid in fat depletion, as well as to enhance anabolic/anticatabolic actions on protein metabolism, said composition consisting essentially of:

| | |
|---|---|
| Ornithine Alpha-ketoglutarate | 1000 mg |
| Potassium Bicarbonate | 100 mg |
| Sodium Bicarbonate | 100 mg |
| Glycine | 100 mg |
| Isopropyl Myristate | 800 mg |
| Distilled Water | 700 mg |
| Vitamin Skin Smoother with Corn | 2000 mg |

8. The method as claimed in claim 7, wherein, the composition is administered topically.
